(19) United States
(12) Patent Application Publication
(10) Pub. No.: US 2013/0244935 A1
(43) Pub. Date: Sep. 19, 2013

(54) Methods of Treating Necrotizing Enterocolitis Using Heparin Binding Epidermal Growth Factor

(71) Applicant: Gail E. Besner, Dublin, OH (US)

(72) Inventor: Gail E. Besner, Dublin, OH (US)

(73) Assignee: NATIONWIDE CHILDREN'S HOSPITAL, INC., COLUMBUS, OH (US)

(21) Appl. No.: 13/793,940

(22) Filed: Mar. 11, 2013

Related U.S. Application Data
(63) Continuation of application No. 13/123,420, filed on Jul. 5, 2011, now abandoned.
(60) Provisional application No. 61/104,515, filed on Oct. 10, 2008.

(51) Int. Cl. A61K 38/17

(52) U.S. Cl. CPC A61K 38/177
USPC 514/9.6

(57) ABSTRACT

Methods of treating, abating and reducing the lisk for necrotizing enterocolitis (NEC) in an infant are disclosed. Preferred methods include admmistering an EGF receptor agonist, such as HB-EGF or EGF, within 24 hours following birth or following the onset of at least one symptom of NEC, in an amount effective to reduce the onset or seventy of NEC.

This application claims priority benefit of U.S. Provisional Patent Application No. 61/104,515, filed Oct. 10, 2008, which is incorporated by reference herein in its entirety.

### FIELD OF INVENTION

The invention provides for methods of treating, abating and reducing the risk for necrotizing enterocolitis (NEC) in an infant by administering an EGF receptor agonist, such as HB-EGF or EGF, within 24 hours following birth or within 24 hours following onset of at least one symptom of NEC, in an amount effective to reduce the onset or severity of NEC.

### BACKGROUND

Necrotizing enterocolitis (NEC) is the most common gastrointestinal emergency in premature newborn infants (Schnabl et al., World J Gastroenterol 14:2142-2161, 2008; Kliegman et al., N Engl J Med 310:1093-103, 1984). With aggressive management leading to the salvage of premature infants from the pulmonary standpoint, the incidence of NEC is increasing, and it is thought that NEC will soon replace pulmonary insufficiency as the leading cause of death in premature infants (Lee et al., Semin Neonatol 8:449-59, 2003). The mortality of this disease ranges from 20% to 50%, resulting in over 1000 infant deaths in this country each year (Caplan et al., Pediatr 13: 111-115, 2001) Like other diseases manifested by severe intestinal injury, NEC can cause the dysregulated inflammation characteristic of the systemic inflammatory response syndrome (SIRS), potentially resulting in multiple organ dysfunction syndrome (MODS) and death. Evidence suggests that the risk factors for NEC, namely formula feeding, intestinal ischemia and bacterial colonization, stimulate proinflammatory mediators that in turn activate a series of events culminating in necrosis of the bowel (Caplan et al., Pediatr 13: 111-115, 2001). Survivors of acute NEC frequently develop malabsorption, malnutrition, total parenteral nutrition-related complications, intestinal strictures and short bowel syndrome (Caplan et al., Pediatr 13:111-115, 2001).

Since prematurity is the single most important risk factor for NEC, it is possible that absent or reduced levels of specific factors that are normally expressed during later periods of gestation may contribute to the development of this condition. With this in mind, exogenous replacement of key factors may be clinically valuable as a means to reduce the incidence of NEC. Several potential preventive strategies have aimed at induction of gastrointestinal maturation with steroids, improvement in host defense with breast milk feeding or oral immunoglobulins, change in bacterial colonization with antibiotics, probiotics or feeding modifications, and reduction or antagonism of inflammatory mediators, none of which have led to consistently positive therapeutic results (Feng et al., Semin Pediatr Surg 14:167-74, 2005).

Heparin-binding epidermal growth factor (HB-EGF) was first identified in the conditioned medium of cultured human macrophages and later found to be a member of the epidermal growth factor (EGF) family of growth factors (Higashiyama et al., Science. 251:936-9, 1991). It is synthesized as a transmembrane, biologically active precursor protein (proHB-EGF) composed of 208 amino acids, which is enzymatically cleaved by matrix metalloproteinases (MMPs) to yield a 14-20 kDa soluble growth factor (sHB-EGF). Pro-HB-EGF can form complexes with other membrane proteins including CD9 and integrin α3β1; these binding interactions function to enhance the biological activity of pro-HB-EGF. ProHB-EGF is a juxtacrine factor that can regulate the function of adjacent cells through its engagement of cell surface receptor molecules.

Like other family members, HB-EGF binds to the EGF receptor (EGFR; ErbB-1), inducing its phosphorylation. Unlike most EGF family members, HB-EGF has the ability to bind strongly to heparan. Cell-surface heparan-sulfate proteoglycans (HSPG) can act as low affinity, high capacity receptors for HB-EGF. HB-EGF is produced by many different cell types including epithelial cells, and it is mitogenic and chemotactic for smooth muscle cells, keratinocytes, hepatocytes and fibroblasts. HB-EGF exerts its mitogenic effects by binding and activation of EGF receptor subtypes ErbB-1 and ErbB-4 (Junttila et al., Trends Cardiovasc Med; 10:304-310, 2001).

However, while the mitogenic function of HB-EGF is mediated through activation of ErbB-1, its migration-inducing function involves the activation of ErbB-4 and the more recently described N-arginine dibasic convertase (NRDc, Nardilysin). This is in distinction to other EGF family members, such as EGF itself, transforming growth factor (TGF)-α and amphiregulin (AR), which exert their signal-transducing effects via interaction with ErbB-1 only. In fact, the NRDc receptor is completely HB-EGF-specific. The differing affinities of EGF family members for the different EGFR subtypes and for HSPG may confer different functional capabilities to these molecules in vivo. The combined interactions of HB-EGF with HSPG and ErbB-1/ErbB-4/NRDc may confer a functional advantage to this growth factor. Importantly, endogenous HB-EGF is protective in various pathologic conditions and plays a pivotal role in mediating the earliest cellular responses to proliferative stimuli and cellular injury.

Administration of EGF to prevent tissue damage after an ischemic event in the brains of gerbils has been reported in U.S. Pat. No. 5,057,494 issued Oct. 15, 1991 to Sheffield. The patent projects that EGF “analogs” having greater than 50% homology to EGF may also be useful in preventing tissue damage and that treatment of damage in myocardial tissue, renal tissue, spleen tissue, intestinal tissue, and lung tissue with EGF or EGF analogs may be indicated. However, the patent includes no experimental data supporting such projections.

The small intestine receives the majority of its blood supply from the superior mesenteric artery (SMA), but also has a rich collateral network such that only extensive perturbations of blood flow lead to pathologic states. VIIIa et al. (Gastroenterology, 110(4 Suppl): A372, 1996) reports that in a rat model of intestinal ischemia in which thirty minutes of ischemia are caused by occlusion of the SMA, pre-treatment of the intestines with EGF attenuated the increase in intestinal permeability compared to that in untreated rats. The intestinal permeability increase is an early event in intestinal tissue changes during ischemia. Multiple animal models, like that described in VIIIa et al., supra have been used to study the effects of ischemic injury to the small bowel. Since the small intestine has such a rich vascular supply, researchers have used complete SMA occlusion to study ischemic injury of the bowel. Animals that experience total SMA occlusion for long periods of time suffer from extreme fluid loss and uniformly die from hypovolemia and sepsis, making models of this type useless for evaluating the recovery from intestinal ischemia. Nevertheless, the sequence of morphologic and physiologic changes in the intestines resulting from ischemic injury has remained an area of intense examination.

Miyazaki et al., Biochem Biophys Res Comm, 226: 542-546 (1996) discusses the increased expression in a rat gastric mucosal cell line of HB-EGF and AR resulting from oxidative stress. The authors speculate that the two growth factors may trigger the series of reparative events following acute injury (apparently ulceration) of the gastrointestinal tract.

EGF family members are of interest as intestinal protective agents due to their roles in gut maturation and function. Infants with NEC have decreased levels of salivary EGF, as do very premature infants (Shin et al., J Pediatr Surg 35:173-176, 2000; Warner et al., J Pediatr 150:358-6, 2007). Studies have demonstrated the importance of EGF in preserving gut barrier function, increasing intestinal enzyme activity, and improving nutrient transport (Warner et al., Semin Pediatr Surg 14:175-80, 2005). EGF receptor (EGFR) knockout mice develop epithelial cell abnormalities and hemorrhagic necrosis of the intestine similar to neonatal NEC, suggesting that lack of EGFR stimulation may play a role in the development of NEC (Miettinen et al., Nature 376:337-41, 1995). Dvorak et al. have shown that EGF supplementation reduces the incidence of experimental NEC in rats, in part by reducing apoptosis, barrier failure, and hepatic dysfunction (Am J Physiol Gastrointest Liver Physiol 282:G156-G164, 2002). Vinter-Jensen et al., investigated the effect of subcutaneously administered EGF (150 μg/kg/12 hours) in rats, for 1, 2 and 4 weeks, and found that EGF induced growth of small intestinal mucosa and muscularis in a time-dependent manner (Regul Pept 61:135-142, 1996). Several case reports of clinical administration of EGF also exist. Sigalet et al. administered EGF (100 μg/kg/day) mixed with enteral feeds for 6 weeks to pediatric patients with short bowel syndrome (SBS), and reported improved nutrient absorption and increased tolerance to enteral feeds with no adverse effects (J Pediatr Surg 40:763-8, 2005). Sullivan et al., in a prospective, double-blind, randomized controlled study that included 8 neonates with NEC, compared the effects of a 6-day continuous intravenous infusion of EGF (100 ng/kg/hour) to placebo, and found a positive trophic effect of EGF on the intestinal mucosa (Ped Surg 42:462-469, 2007). Palomino et al. examined the efficacy of EGF in the treatment of duodenal ulcers in a multicenter, randomized, double blind human clinical trial in adults. Oral human recombinant EGF (50 mg/ml every 8 h for 6 weeks) was effective in the treatment of duodenal ulcers with no side effects noted (Stand J Gastroenterol 35:1016-22, 2000).

Enteral administration of E. coli-derived HB-EGF has been shown to decrease the incidence and severity of intestinal injury in a neonatal rat model of NEC, with the greatest protective effects found at doses of 600 or 800 μg/kg/dose (Feng et al., Semin Pediatr Surg 14:167-74, 2005). In addition, HB-EGF is known to protect the intestines from injury after intestinal ischemia/reperfusion injury (El-Assal et al., Semin Pediatr Surg 13:2-10, 2004) or hemorrhagic shock and resuscitation (El-Assal et al., Surgery 142:234-42, 2007).

The prevention and treatment of ischemic damage in the clinical setting continues to be a challenge in medicine. There exists a need in the art for models for testing the effects of potential modulators of ischemic events and for methods of preventing and/or treating ischemic damage, particularly ischemic damage to the intestines. Because of its ability to enhance the regenerative capacity and/or increase the resistance of the mucosa to injury, HB-EGF may represent a promising therapeutic strategy for intestinal diseases, including necrotizing enterocolitis.

### SUMMARY OF INVENTION

HB-EGF is known to be present in human amniotic fluid and breast milk, ensuring continuous exposure of the fetal and newborn intestine to endogenous levels of the growth factor (Michalsky et al., J Pediatr Surg 37:1-6, 2006). Thus, the developing fetus and the breastfed newborn are continually exposed to HB-EGF naturally both before and after birth. Supplementation of enteral feeds with a biologically active substance such as HB-EGF, to which the fetus and newborn are naturally exposed, may represent a logical and safe way to reduce intestinal injury resulting in NEC. HB-EGF supplementation of feeds in very low birth weight (VLBW) patients (<1500 g) who are most at risk for developing NEC is contemplated to facilitate maturation, enhance regenerative capacity, and increase the resistance of the intestinal mucosa to injury.

Intragastric administration of HB-EGF to rats is known to lead to delivery of the growth factor to the entire GI tract including the colon within 8 hours. HB-EGF is excreted in the bile and urine after intragastric or intravenous administration (Feng et al., Peptides. 27(6):1589-96, 2006). In addition, intragastric administration of HB-EGF to neonatal rats and minipigs has no systemic absorption of the growth factor (unpublished data). These findings collectively support the clinical feasibility and safety of enteral administration of HB-EGF in protection of the intestines from injury.

The invention provides for methods of treating an infant suffering from or at risk for necrotizing enterocolitis (NEC), comprising administering an EGF receptor agonist in an amount effective to reduce the onset or severity of NEC, wherein the EGF receptor agonist is administered within about 24 hours following birth.

The invention also provides for methods of treating an infant to abate necrotizing enterocolitis (NEC) in an infant, comprising administering an EGF receptor agonist in an amount effective to reduce the onset of NEC or severity of NEC, wherein the EGF receptor agonist is administered within about 24 hours following birth.

In a further embodiment, the invention provides for methods of reducing the risk of developing necrotizing enterocolitis (NEC) in an infant, comprising administering an EGF receptor agonist in an amount effective to reduce the onset of NEC, wherein the EGF receptor agonist is administered within about 24 hours following birth.

In another embodiment, the invention provides for methods of treating an infant suffering from or at risk for necrotizing enterocolitis (NEC), comprising administering an EGF receptor agonist in an amount effective to reduce the onset or severity of NEC, wherein the EGF receptor agonist is administered within about 24 hours following onset of at least one symptom of NEC.

The onset of symptoms of NEC refers to the occurrence or presence of one or more of the following symptoms: temperature instability, lethargy, apnea, bradycardia, poor feeding, increased pregavage residuals, emesis (may be bilious or test positive for occult blood), abdominal distention (mild to marked), occult blood in stool (no fissure), gastrointestinal bleeding (mild bleeding to marked hemorrhaging), significant intestinal distention with ileus, small-bowel separation, edema in bowel wall or peritoneal fluid, unchanging or persistent “rigid” bowel loops, pneumatosis intestinalls, portal venous gas, deterioration of vital signs, evidence of septic shock and pneumoperitoneum.

In one embodiment, the invention contemplates administering an EGF receptor agonist to a premature infant. The term “premature infant” (also known as a “premature baby” or a “preemie”) refers to babies born having less than 36 weeks gestation. In another embodiment, the invention provides for methods of administering an EGF receptor agonist to an infant having a low birth weight or a very low birth weight. A low birth weight is a weight less than 2500 g (5.5 lbs.). A very low birth weight is a weight less than 1500 g (about 3.3 lbs.). The invention also provides for methods of administering HB-EGF to infants having intrauterine growth retardation, fetal alcohol syndrome, drug dependency, prenatal asphyxia, shock, sepsis, or congenital heart disease.

The methods of the invention may utilize any EGF receptor agonist. An EGF receptor agonist refers to a molecule or compound that activates the EGF receptor or induces the EGF receptor to dimerize, autophosphorylate and initiate cellular signaling. For example, any of the methods of the invention may be carried out with an EGF receptor agonist such as an EGF product or an HB-EGF product.

The methods of the invention are carried out with a dose of an EGF receptor agonist that is effective to reduce the onset or severity of NEC. Exemplary effective doses are 100 μg/kg dose, 105 μg/kg dose, 110 μg/kg dose, 115 μg/kg dose, 120 μg/kg dose, 125 μg/kg dose, 130 μg/kg dose, 135 μg/kg dose, 140 μg/kg dose, 200 jag/kg dose, 250 μg/kg dose, 300 μg/kg dose, 400 μg/kg dose, 500 μg/kg dose, 550 μg/kg dose, 570 μg/kg dose, 600 μg/kg dose, 800 μg/kg dose and 1000 μg/kg dose. Exemplary dosage ranges of EGF receptor agonist that is effective to reduce the onset or severity of NEC are 100-140 μg/kg, 100-110 μg/kg dose, 110-120 μg/kg dose, 120-130 μg/kg dose, 120-140 μg/kg dose and 130-140 μg/kg dose For example, the dose may be administered within about the first hour following birth, within about 2 hours following birth, within about 3 hours following birth, within about 4 hours following birth, within about 5 hours following birth, within about 6 hours following birth, within about 7 hours following birth, within about 8 hours following birth, within about 9 hours following birth, within about 10 hours following birth, within about 11 hours following birth, within about 12 hours after birth, within about 13 hours after birth, within about 14 hours after birth, within about 15 hours after birth, within about 16 hours after birth, within about 17 hours after birth, within about 18 hours after birth, within about 19 hours after birth, within about 20 hours after birth, within about 21 hours after birth, within about 22 hours after birth, within about 23 hours after birth, within about 24 hours after birth, within about 36 hours after birth, within about 48 hours after birth or within about 72 hours after birth.

The invention contemplates administering an EGF receptor agonist to an infant suffering or at risk of developing NEC. In one embodiment, an EGF receptor agonist is administered within about the first 12-72 hours after birth. For example, the dose of an EGF receptor agonist may be administered about 12 hours after birth, about 24 hours after birth, about 36 hours after birth, about 48 hours after birth or about 72 hours after birth. In further embodiments, the dose may be administered between hours 1-4 following birth or between hours 2-5 following birth or between hours 3-6 following birth or between hours 4-7 following birth or between hours 5-8 following birth or between hours 6-9 following birth or between hours 7-10 following birth or between hours 8-11 following birth, between hours 9-12 following birth, between hours 10-13 following birth, between hours 11-14 following birth, between hours 12-15 following birth, between hours 13-16 following birth, between hours 14-17 following birth, between hours 15-18 following birth, between hours 16-19 following birth, between hours 17-20 following birth, between hours 18-21 following birth, between hours 19-22 following birth, between hours 20-23 following birth, between hours 21-24 following birth, between hours 12-48 following birth, between hours 24-36 following birth, between hours 36-48 following birth and between hours 48-72 after birth

In another embodiment, an EGF receptor agonist is administered within 24 hours following the onset of at least one symptom of NEC, such as administering an EGF receptor agonist within about the first 12-72 hours after onset of at least one symptom of NEC. For example, the dose of an EGF receptor agonist may be administered about 12 hours following the occurrence or presence of a symptom of NEC, about 24 hours following the occurrence or presence of a symptom of NEC, about 36 hours following the occurrence or presence of a symptom of NEC, about 48 hours following the occurrence or presence of a symptom of NEC or about 72 hours following the occurrence or presence of a symptom of NEC. In further embodiments, the dose may be administered between hours 1-4 following the occurrence or presence of a symptom of NEC or between hours 2-5 following the occurrence or presence of a symptom of NEC or between hours 3-6 following the occurrence or presence of a symptom of NEC or between hours 4-7 following the occurrence or presence of a symptom of NEC or between hours 5-8 following the occurrence or presence of a symptom of NEC or between hours 6-9 following the occurrence or presence of a symptom of NEC or between hours 7-10 following the occurrence or presence of a symptom of NEC or between hours 8-11 following the occurrence or presence of a symptom of NEC, between hours 9-12 following the occurrence or presence of a symptom of NEC, between hours 10-13 following the occurrence or presence of a symptom of NEC, between hours 11-14 following the occurrence or presence of a symptom of NEC, between hours 12-15 following the occurrence or presence of a symptom of NEC, between hours 13-16 following the occurrence or presence of a symptom of NEC, between hours 14-17 following the occurrence or presence of a symptom of NEC, between hours 15-18 following the occurrence or presence of a symptom of NEC, between hours 16-19 following the occurrence or presence of a symptom of NEC, between hours 17-20 following the occurrence or presence of a symptom of NEC, between hours 19-22 following the occurrence or presence of a symptom of NEC, between hours 20-23 following the occurrence or presence of a symptom of NEC, between hours 21-24 following the occurrence or presence of a symptom of NEC, between hours 12-48 following the occurrence or presence of a symptom of NEC, between hours 24-36 following after the occurrence or presence of a symptom of NEC, between hours 36-48 following the occurrence or presence of a symptom of NEC or between hours 48-72 following the occurrence or presence of a symptom of NEC.

The term “within 24 hours after birth” refers to administering at least a first unit dose of an EGF receptor agonist within about 24 hours following birth, and the first dose may be succeeded by subsequent dosing outside the initial 24 hour dosing period.

The term “within 24 hours following the onset of at least one symptom of NEC” refers to administering at least a first unit dose of an EGF receptor agonist within about 24 hours following the first clinical sign or symptom of NEC. The first dose may be succeeded by subsequent dosing outside the initial 24 hour dosing period.

The EGF receptor agonist may be administered to an infant once a day (QD), twice a day (BID), three times a day (TID), four times a day (QID), five times a day (FID), six times a day (HID), seven times a day or 8 times a day. The EGF receptor agonist may be administered alone or in combination with feeding. The EGF receptor agonist may be administered to an infant with formula or breast milk with every feeding or a portion of feedings.

The methods of the invention may be carried out with any HB-EGF product including recombinant HB-EGF produced in E. coli and HB-EGF produced in yeast. The development of expression systems for the production of recombinant proteins is important for providing a source of protein for research and/or therapeutic use. Expression systems have been developed for both prokaryotic cells such as E. coli, and for eukaryotic cells such as yeast (Saccharomyces, Pichia and Kluyveromyces spp) and mammalian cells.

### EGF Receptor Agonists

The Epidermal Growth Factor Receptor (EGFR) is a transmembrane glycoprotein that is a member of the protein kinase superfamily. The EGFR is a receptor for members of the epidermal growth factor family. Binding of the protein to a receptor agonist induces receptor dimerization and tyrosine autophosphorylation, and leads to cell proliferation and various other cellular effects (e.g. chemotaxis, cell migration).

The amino acid sequence of the EGF receptor is set out as SEQ ID NO: 16 (Genbank Accession No. NP_{—}005219). EGF receptors are encoded by the nucleotide sequence set out as SEQ ID NO: 15 (Genbank Accession No. NM_{—}005228). The EGF receptor is also known in the art as EGFR, ERBB, HER1, mENA, and PIG61. An EGF receptor agonist is a molecule that binds to and activates the EGF receptor so that the EGF receptor dimerizes with the appropriate partner and induces cellular signaling and ultimately results in an EGF receptor-induced biological effect, such as cell proliferation, cell migration or chemotaxis. Exemplary EGF receptor agonists include epidermal growth factor (EGF), heparin binding EGF (HB-EGF), transforming growth factor-α (TGF-α), amphiregulin, betacellulin, epiregulin, and epigen.

### Epidermal Growth Factor

Epidermal Growth Factor (EGF), also known as beta-urogastrone, URG and HOMG4, is a potent mitogenic and differentiation factor. The amino acid sequence of EGF is set out as SEQ ID NO: 4 (Genbank Accession No. NP_{—}001954). EGF is encoded by the nucleotide sequence set out as SEQ ID NO: 3 (Genbank Accession No. NM_{—}001963).

As used herein, “EGF product” includes EGF proteins comprising about amino acid 1 to about amino acid 1207 of SEQ ID NO: 4; EGF proteins comprising about amino acid 1 to about amino acid 53 of SEQ ID NO: 4; fusion proteins comprising the foregoing EGF proteins; and the foregoing EGF proteins including conservative amino acid substitutions. In a specific embodiment, the EGF product is human EGF(1-53), which is a soluble active polypeptide. Conservative amino acid substitutions are understood by those skilled in the art. The EGF products may be isolated from natural sources, chemically synthesized, or produced by recombinant techniques. In order to obtain EGF products of the invention, EGF precursor proteins may be proteolytically processed in situ. The EGF products may be post-translationally modified depending on the cell chosen as a source for the products.

The EGF products of the invention are contemplated to exhibit one or more biological activities of EGF, such as those described in the experimental data provided herein or any other EGF biological activity known in the art. For example, the EGF products of the invention may exhibit one or more of the following biological activities: cellular mitogenicity in a number of cell types including epithelial cells and smooth muscle cells, cellular survival, cellular migration, cellular differentiation, organ morphogenesis, epithelial cytoprotection, tissue tropism, cardiac function, wound healing, epithelial regeneration, promotion of hormone secretion such as prolactin and human gonadotrophin, pituitary hormones and steroids, and influence glucose metabolism.

The present invention provides for the EGF products encoded by the nucleic acid sequence of SEQ ID NO: 4 or fragments thereof including nucleic acid sequences that hybridize under stringent conditions to the complement of the nucleotides sequence of SEQ ID NO: 3, a polynucleotide which is an allelic variant of SEQ ID NO: 3; or a polynucleotide which encodes a species homolog of SEQ ID NO: 4.

### HB-EGF Polypeptide

The cloning of a cDNA encoding human HB-EGF (or HB-EHM) is described in Higashiyama et al., Science, 251: 936-939 (1991) and in a corresponding international patent application published under the Patent Cooperation Treaty as International Publication No. WO 92/06705 on Apr. 30, 1992. Both publications are hereby incorporated by reference herein in their entirety. In addition, uses of human HB-EGF are taught in U.S. Pat. No. 6,191,109 and International Publication No. WO 2008/134635(Intl. Appl. No. PCT/US08/61772), also incorporated by reference in its entirety.

The sequence of the protein coding portion of the cDNA is set out in SEQ ID NO: 1 herein, while the deduced amino acid sequence is set out in SEQ ID NO: 2. Mature HB-EGF is a secreted protein that is processed from a transmembrane precursor molecule (pro-HB-EGF) via extracellular cleavage. The predicted amino acid sequence of the full length HB-EGF precursor represents a 208 amino acid protein. A span of hydrophobic residues following the translation-initiating methionine is consistent with a secretion signal sequence. Two threonine residues (Thr75 and Thr85 in the precursor protein) are sites for O-glycosylation. Mature HB-EGF consists of at least 86 amino acids (which span residues 63-148 of the precursor molecule), and several microheterogeneous forms of HB-EGF, differing by truncations of 10, 11, 14 and 19 amino acids at the N-terminus have been identified. HB-EGF contains a C-terminal EGF-like domain (amino acid residues 30 to 86 of the mature protein) in which the six cysteine residues characteristic of the EGF family members are conserved and which is probably involved in receptor binding. HB-EGF has an N-terminal extension (amino acid residues 1 to 29 of the mature protein) containing a highly hydrophilic stretch of amino acids to which much of its ability to bind heparin is attributed. Besner et al., Growth Factors, 7: 289-296 (1992), which is hereby incorporated by reference herein, identifies residues 20 to 25 and 36 to 41 of the mature HB-EGF protein as involved in binding cell surface heparin sulfate and indicates that such binding mediates interaction of HB-EGF with the EGF receptor.

As used herein, “HB-EGF product” includes HB-EGF proteins comprising about amino acid 63 to about amino acid 148 of SEQ ID NO: 2 (HB-EGF(63-148)); HB-EGF proteins comprising about amino acid 73 to about amino acid 148 of SEQ ID NO: 2 (HB-EGF(73-148)); HB-EGF proteins comprising about amino acid 74 to about amino acid 148 of SEQ ID NO: 2 (HB-EGF(74-148)); HB-EGF proteins comprising about amino acid 77 to about amino acid 148 of SEQ ID NO: 2 (HB-EGF(77-148)); HB-EGF proteins comprising about amino acid 82 to about amino acid 148 of SEQ ID NO: 2 (HB-EGF(82-148)); HB-EGF proteins comprising a continuous series of amino acids of SEQ ID NO: 2 which exhibit less than 50% homology to EGF and exhibit HB-EGF biological activity, such as those described herein; fusion proteins comprising the foregoing HB-EGF proteins; and the foregoing HB-EGF proteins including conservative amino acid substitutions. In a specific embodiment, the HB-EGF product is human HB-EGF(74-148). Conservative amino acid substitutions are understood by those skilled in the art. The HB-EGF products may be isolated from natural sources known in the art (e.g., the U-937 cell line (ATCC CRL 1593)), chemically synthesized, or produced by recombinant techniques such as disclosed in WO92/06705, supra, the disclosure of which is hereby incorporated by reference. In order to obtain HB-EGF products of the invention, HB-EGF precursor proteins may be proteolytically processed in situ. The HB-EGF products may be post-translationally modified depending on the cell chosen as a source for the products.

The HB-EGF products of the invention are contemplated to exhibit one or more biological activities of HB-EGF, such as those described in the experimental data provided herein or any other HB-EGF biological activity known in the art. One such biological activity is that HB-EGF products compete with HB-EGF for binding to the ErbB-1 receptor and has ErbB-1 agonist activity. In addition, the HB-EGF products of the invention may exhibit one or more of the following biological activities: cellular mitogenicity, cellular chemoattractant, endothelial cell migration, acts as a pro-survival factor (protects against apoptosis), decrease inducible nitric oxide synthase (iNOS) and nitric oxide (NO) production in epithelial cells, decrease nuclear factor-KB (NF-KB) activation, increase eNOS (endothelial nitric oxide synthase) and NO production in endothelial cells, stimulate angiogenesis and promote vasodilatation.

The present invention provides for the HB-EGF products encoded by the nucleic acid sequence of SEQ ID NO: 1 or fragments thereof including nucleic acid sequences that hybridize under stringent conditions to the complement of the nucleotides sequence of SEQ ID NO: 1, a polynucleotide which is an allelic variant of any SEQ ID NO: 1; or a polynucleotide which encodes a species homolog of SEQ ID NO: 2.

### Additional EGF Receptor Agonists

Additional EGF receptor agonists include: Transforming Growth Factor-α (TGF-α), also known as TFGA, which has the amino acid sequence set out as SEQ ID NO: 6 (Genbank Accession No. NP_{—}001093161), and is encoded by the nucleotide sequence set out as SEQ ID NO: 5 (Genbank Accession No. NM_{—}001099691); amphiregulin, also known as AR, SDGF, CRDGF, and MGC13647, which has the amino acid sequence set out as SEQ ID NO: 8 (Genbank Accession No. NP_{—}001648), and is encoded by the nucleotide sequence set out as SEQ ID NO: 7 (Genbank Accession No. NM_{—}001657); betacellulin (BTG) which has the amino acid sequence set out as SEQ ID NO: 10 (Genbank Accession No. NP_{—}001720), and is encoded by the nucleotide sequence set out as SEQ ID NO: 9 (Genbank Accession No. NM_{—}001729); Epiregulin (EREG), also known as ER, which has the amino acid sequence set out as SEQ ID NO: 12 (Genbank Accession No. NP_{—}001423) and is encoded by the nucleotide sequence set out as SEQ ID NO: 11 (Genbank Accession No. NM_{—}001432); and epigen (EPGN) also known as epithelial mitogen homolog, EPG, PRO9904, ALGV3072, FLJ75542, which has the amino acid sequence set out as SEQ ID NO: 14 (Genbank Accession No. NP_{—}001013460), and is encoded by the nucleotide sequence set out as SEQ ID NO: 13 (Genbank Accession No. NM_{—}001013442).

The EGF receptor agonists also may be encoded by nucleotide sequences that are substantially equivalent to any of the EGF receptor agonists polynucleotides recited above. Polynucleotides according to the invention can have at least, e.g., 65%, 70%, 75%, 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to the polynucleotides recited above. Preferred computer program methods to determine identity and similarity between two sequences include, but are not limited to, the GCG program package, including GAP (Devereux et al., Nucl. Acid. Res., 12: 387, 1984; Genetics Computer Group, University of Wisconsin, Madison, Wis.), BLASTP, BLASTN, and FASTA (Altschul et al., J. Mol. Biol., 215: 403-410, 1990). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

Included within the scope of the nucleic acid sequences of the invention are nucleic acid sequence fragments that hybridize under stringent conditions to any of SEQ ID NOS: 1, 3, 5, 7, 9, 11 and 13, or compliments thereof, which fragment is greater than about 5 nucleotides, preferably 7 nucleotides, more preferably greater than 9 nucleotides and most preferably greater than 17 nucleotides. Fragments of, e.g., 15, 17, or 20 nucleotides or more that are selective for (i.e., specifically hybridize to any one of the polynucleotides of the invention) are contemplated.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989). More stringent conditions (such as higher temperature, lower ionic strength, higher formamide, or other denaturing agent) may also be used, however, the rate of hybridization will be affected. In instances wherein hybridization of deoxyoligonucleotides is concerned, additional exemplary stringent hybridization conditions include washing in 6×SSC 0.05% sodium pyrophosphate at 37° C. (for 14-base oligos), 48° C. (for 17-base oligos), 55° C. (for 20-base oligos), and 60° C. (for 23-base oligos).

Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO₄, (SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: A Practical Approach, Ch. 4, IRL Press Limited (Oxford, England). Hybridization conditions can be adjusted by one skilled in the art in order to accommodate these variables and allow DNAs of different sequence relatedness to form hybrids.

The EGF receptor agonists of the invention include, but are not limited to, a polypeptide comprising: the amino acid sequences encoded by the nucleotide sequence of any one of SEQ ID NOS: 1, 3, 5, 7, 9, 11 and 13, or the corresponding full length or mature protein. In one embodiment, polypeptides of the invention also include polypeptides preferably with EGF receptor agonist biological activity described herein that are encoded by: (a) an open reading frame contained within any one of the nucleotide sequences set forth as SEQ ID NO: 1, 3, 5, 7, 9, 11 and 13, preferably the open reading frames therein or (b) polynucleotides that hybridize to the complement of the polynucleotides of (a) under stringent hybridization conditions. In another embodiment, polypeptides of the invention also include polypeptides preferably with EGF receptor agonist biological activity described herein that are encoded by: (a) an open reading frame contained within the nucleotide sequences set forth any as SEQ ID NO: 1, 3, 5, 7, 9, 11 and 13, preferably the open reading frames therein or (b) polynucleotides that hybridize to the complement of the polynucleotides of (a) under stringent hybridization conditions.

The EGF receptor agonists of the invention also include biologically active variants of any of the amino acid sequences of SEQ ID NO: 2, 4, 6, 8, 10, 12 and 14; and “substantial equivalents” thereof with at least, e.g., about 65%, about 70%, about 75%, about 80%, about 85%, 86%, 87%, 88%, 89%, at least about 90%, 91%, 92%, 93%, 94%, typically at least about 95%, 96%, 97%, more typically at least about 98%, or most typically at least about 99% amino acid identity) that retain EGF receptor agonist biological activity. Polypeptides encoded by allelic variants may have a similar, increased, or decreased activity compared to polypeptides having the amino acid sequence of any of SEQ ID NO: 2, 4, 6, 8, 10, 12 and 14.

The EGF receptor agonists of the invention include polypeptides with one or more conservative amino acid substitutions that do not affect the biological activity of the polypeptide. Alternatively, the EGF receptor agonist polypeptides of the invention are contemplated to have conservative amino acids substitutions which may or may not alter biological activity. The term “conservative amino acid substitution” refers to a substitution of a native amino acid residue with a normative residue, including naturally occurring and normaturally occurring amino acids, such that there is little or no effect on the polarity or charge of the amino acid residue at that position. For example, a conservative substitution results from the replacement of a non-polar residue in a polypeptide with any other non-polar residue. Further, any native residue in the polypeptide may also be substituted with alanine, according to the methods of “alanine scanning mutagenesis.” Naturally occurring amino acids are characterized based on their side chains as follows: basic: arginine, lysine, histidine; acidic: glutamic acid, aspartic acid; uncharged polar: glutamine, asparagine, serine, threonine, tyrosine; and non-polar: phenylalanine, tryptophan, cysteine, glycine, alanine, valine, proline, methionine, leucine, norleucine, isoleucine.

### Pharmaceutical Compositions

The administration of EGF receptor agonists is preferably accomplished with a pharmaceutical composition comprising an EGF receptor agonist and a pharmaceutically acceptable carrier. The carrier may be in a wide variety of forms depending on the route of administration. Suitable liquid carriers include saline, PBS, lactated Ringer solution, human plasma, human albumin solution, 5% dextrose and mixtures thereof. The route of administration may be oral, rectal, parenteral, or through a nasogastric or orogastric tube (enteral). Examples of parenteral routes of administration are intravenous, intra-arterial, intraperitoneal, intraluminally, intramuscular or subcutaneous injection or infusion.

The presently preferred route of administration of the present invention is the enteral route. Therefore, the present invention contemplates that the acid stability of HB-EGF is a unique factor as compared to, for example, EGF. For example, the pharmaceutical composition of the invention may also include other ingredients to aid solubility, or for buffering or preservation purposes. Pharmaceutical compositions containing EGF receptor agonists may comprise the agonist at a concentration of about 100 to 1000 μg/kg in saline. Suitable doses are in the range from 100-140 μg/kg, or 100-110 μg/kg, or 110-120 μg/kg, or 120-130 μg/kg, or 120-140 μg/kg, or 130-140 μg/kg, or 500-700 μg/kg, or 600-800 μg/kg or 800-1000 μg/kg. Preferred doses include 100 μg/kg, 120 μg/kg, 140 μg/kg and 600 μg/kg administered enterally once a day. Additional preferred doses may be administered once, twice, three, four, five, six or seven or eight times a day enterally.

The dose of EGF receptor agonist may also be administered intravenously. In addition, the dose of EGF receptor agonist may be administered as a bolus, either once at the onset of therapy or at various time points during the course of therapy, such as every four hours, or may be infused for instance at the rate of about 0.01 μg/kg/h to about 5 μg/kg/h during the course of therapy until the patient shows signs of clinical improvement. Addition of other bioactive compounds [e.g., antibiotics, free radical scavenging or conversion materials (e.g., vitamin E, beta-carotene, BHT, ascorbic acid, and superoxide dimutase), fibrolynic agents (e.g., plasminogen activators), and slow-release polymers] to the EGF receptor agonist or separate administration of the other bioactive compounds is also contemplated.

As used herein, “pathological conditions associated with intestinal ischemia” includes conditions which directly or indirectly cause intestinal ischemia (e.g., premature birth, birth asphyxia, congenital heart disease, cardiac disease, polycythemia, hypoxia, exchange transfusions, low-flow states, atherosclerosis, embolisms or arterial spasms, ischemia resulting from vessel occlusions in other segments of the bowel, ischemic colitis, and intestinal torsion such as occurs in infants and particularly in animals) and conditions which are directly or indirectly caused by intestinal ischemia (e.g., necrotizing enterocolitis, shock, sepsis, and intestinal angina). Thus, the present invention contemplates administration of an EGF receptor agonist to patients in need of such treatment including patients at risk for intestinal ischemia, patients suffering from intestinal ischemia, and patients recovering from intestinal ischemia. The administration of an EGF receptor agonist to patients is contemplated in both the pediatric and adult populations.

More particularly, the invention contemplates a method of reducing necrosis associated with intestinal ischemia comprising administering an EGF receptor agonist, such as an HB-EGF product or an EGF product, to a patient at risk for, suffering from, or recovering from intestinal ischemia. Also contemplated is a method of protecting intestinal epithelial cells from hypoxia comprising exposing the cells to an HB-EGF product. Administration of, or exposure to, HB-EGF products reduces lactate dehyrogenase efflux from intestinal epithelial cells, maintains F-actin structure in intestinal epithelial cells, increases ATP levels in intestinal epithelial cells, and induces proliferation of intestinal epithelial cells.

In view of the efficacy of HB-EGF in protecting intestinal tissue from ischemic events, it is contemplated that HB-EGF has a similar protective effect on myocardial, renal, spleen, lung, brain and liver tissue.

### Administration to Pediatric Patients

Intestinal injury related to an ischemic event is a major risk factor for neonatal development of necrotizing enterocolitis (NEC). NEC accounts for approximately 15% of all deaths occurring after one week of life in small premature infants. Although most babies who develop NEC are born prematurely, approximately 10% of babies with NEC are full-term infants. Babies with NEC often suffer severe consequences of the disease ranging from loss of a portion of the intestinal tract to the entire intestinal tract. At present, there are no known therapies to decrease the incidence of NEC in neonates.

Babies considered to be at risk for NEC are those who are premature (less than 36 weeks gestation) or those who are full-term but exhibit, e.g., prenatal asphyxia, shock, sepsis, or congenital heart disease. The presence and severity of NEC is graded using the staging system of Bell et al., J. Ped. Surg., 15:569 (1980) as follows:

| | |
|---|---|
| Stage I | Any one or more historical factors producing perinatal stress |
| (Suspected | Systemic manifestations - temperature instability, lethargy, |
| NEC) | apnea, bradycardia |
| | Gastrointestinal manifestations—poor feeding, increased |
| | pregavage residuals, emesis (may be bilious or test positive |
| | for occult blood), mild abdominal distention, occult blood in |
| | stool (no fissure) |
| Stage II | Any one or more historical factors |
| (Definite | Above signs and symptoms plus persistant occult or gross |
| NEC) | gastrointestinal bleeding, marked abdominal distention |
| | Abdominal radiographs showing significant intestinal |
| | distention with ileus, small-bowel separation (edema in |
| | bowel wall or peritoneal fluid), unchanging or persistent |
| | “rigid” bowel loops, pneumatosis intestinalls, portal venous |
| | gas |
| Stage III | Any one or more historical factors |
| (Advanced | Above sings and symptoms plus deterioration of vital signs, |
| NEC) | evidence of septic shock, or marked gastrointestinal |
| | hemorrhage |
| | Abdominal radiographs showing pneumoperitoneum in |
| | addition to findings listed for Stage II |

Babies at risk for or exhibiting NEC are treated as follows. Patients receive a daily liquid suspension of HB-EGF (e.g. about 1 mg/kg in saline or less). The medications are delivered via a nasogastric or orogastric tube if one is in place, or orally if there is no nasogastric or orogastric tube in place.

### BRIEF DESCRIPTION OF DRAWINGS

FIG. 1A-B depicts analysis of HB-EGF dosing intervals. Panel A shows the NEC Score. The effect of HB-EGF (800 μg/kg/dose) added to feeds two (BID), three (TID), four (QID) or six (HID) times a day on the development of NEC is shown. Each dot represents a single rat pup exposed to experimental NEC, and the NEC score for each pup is shown. Panel B depicts the incidence of NEC. The percent of animals with NEC at each dosing interval is shown. * denotes p<0.05 compared to the non-HB-EGF-treated control group. N/A denotes no addition of HB-EGF to feeds.

FIG. 2A-B depicts the comparison of HB-EGF and EGF in prevention of NEC. Panel A presents NEC scores. Either equal molar (800 mg/kg/dose HB-EGF vs. 570 μg/kg/dose EGF) or equal mass (800 mg/kg/dose HB-EGF vs. 800 mg/kg/dose EGF) amounts of HB-EGF and EGF were compared in their ability to prevent NEC. Each dot represents a single rat pup exposed to experimental NEC, and the NEC score for each pup is shown. Panel B presents the incidence of NEC. The percent of animals with NEC in pups that received either equal molar or equal mass amounts of HB-EGF or EGF is shown. * denotes p<0.05 compared to the non-growth factor-treated control group. N/A denotes no addition of HB-EGF to feeds.

FIG. 3A-B depicts the comparison of prophylactic and therapeutic administration of HB-EGF in NEC. Panel A presents NEC scores. The effect of HB-EGF (800 μg/kg/dose) added to feeds starting with the first feed at 2 h after birth, or at 12, 24, 48 or 72 hours after birth is shown. Each dot represents a single rat pup exposed to experimental NEC, and the NEC score for each pup is shown. Panel B present the incidence of NEC. The percent of animals with NEC in pups that received HB-EGF (800 μg/kg/dose) starting 2, 12, 24, 48 or 72 hours after birth is shown. * denotes p<0.05 compared to the non-HB-EGF-treated control group. N/A denotes no addition of HB-EGF to feeds.

### DETAILED DESCRIPTION

The following examples illustrate the invention wherein Example 1 describes a neonatal rat model of experimental NEC. Example 2 describes experiments relating to dosing intervals for HB-EGF administration. Example 3 describes studies comparing P. pastoris-derived and E. coli-derived HB-EGF. Example 4 describes studies comparing the effect of HB-EGF and EGF in prevention of NEC. Example 5 describes studies comparing prophylactic and therapeutic administration of HB-EGF in the prevention of NEC.

### EXAMPLES

### Example 1

### Neonatal Rat Model of Experimental Necrotizing Enterocolitis

The studies described herein utilize a neonatal rat model of experimental NEC. These experimental protocols were performed according to the guidelines for the ethical treatment of experimental animals and approved by the Institutional Animal Care and Use Committee of Nationwide Children's Hospital (#04203AR). Necrotizing enterocolitis was induced using a modification of the neonatal rat model of NEC initially described by Barlow et al. (J Pediatr Surg 9:587-95, 1974). Pregnant time-dated Sprague-Dawley rats (Harlan Sprague-Dawley, Indianapolis, Ind.) were delivered by C-section under CO₂ anesthesia on day 21.5 of gestation. Newborn rats were placed in a neonatal incubator for temperature control. Neonatal rats were fed via gavage with a formula containing 15 g Similac 60/40 (Ross Pediatrics, Columbus, Ohio) in 75 mL Esbilac (Pet-Ag, New Hampshire, Ill.), a diet that provided 836.8 kJ/kg per day. Feeds were started at 0.1 mL every 4 hours beginning 2 hours after birth and advanced as tolerated up to a maximum of 0.4 mL per feeding by the fourth day of life. Animals were also exposed to a single dose of intragastric lipopolysaccharide (LPS; 2 mg/kg) 8 hours after birth, and were stressed by exposure to hypoxia (100% nitrogen for 1 minute) followed by hypothermia (4° C. for 10 minutes) twice a day beginning immediately after birth and continuing until the end of the experiment. In all experiments, pups were euthanized by cervical dislocation upon the development of any clinical signs of NEC. All remaining animals were sacrificed at the end of experiment at 96 hours after birth.

The HB-EGF used in all experiments was GMP-grade human mature HB-EGF produced in P. pastoris yeast (KBI BioPharma, Inc., Durham, N.C.). EGF was produced in E. coli and purchased from Vybion, Inc. (Ithaca, N.Y.).

To assess the histologic injury score, immediately upon sacrifice, the gastrointestinal tract was carefully removed and visually evaluated for typical signs of NEC including areas of bowel necrosis, intestinal hemorrhage and perforation. Three pieces each of duodenum, jejunum, ileum, and colon from every animal were fixed in 10% formalin for 24 hours, paraffin-embedded, sectioned at 5 μm thickness, and stained with hematoxylin and eosin for histological evaluation of the presence and/or degree of NEC using the NEC histologic injury scoring system described by Caplan et al. (Pediatr Pathol 14:1017-28, 1994). Histological changes in the intestines were graded as follows: grade 0, no damage; grade 1, epithelial cell lifting or separation; grade 2, sloughing of epithelial cells to the mid villus level; grade 3, necrosis of the entire villus; and grade 4, transmural necrosis. All tissues were graded blindly by two independent observers. Tissues with histological scores of 2 or higher were designated as positive for NEC.

Fisher's exact test was used for comparing the incidence of NEC between groups with no adjustments made for multiple comparisons. P-values less then 0.05 were considered statistically significant. All statistical analyses were performed using SAS, (version 9.1, SAS Institute, Cary, N.C.).

### Example 2

### Dosing Interval of HB-EGF Administration

Enteral administration of HB-EGF at doses of 600 or 800 μg/kg/dose administered six times a day is known to significantly decrease the incidence and severity of experimental NEC (Feng et al., Pediatr Surg 41:144-149, 2006). It was of interest to investigate whether administration fewer than six times a day could also protect the intestines from NEC. In particular, the effect of decreasing HB-EGF dosing intervals was investigated.

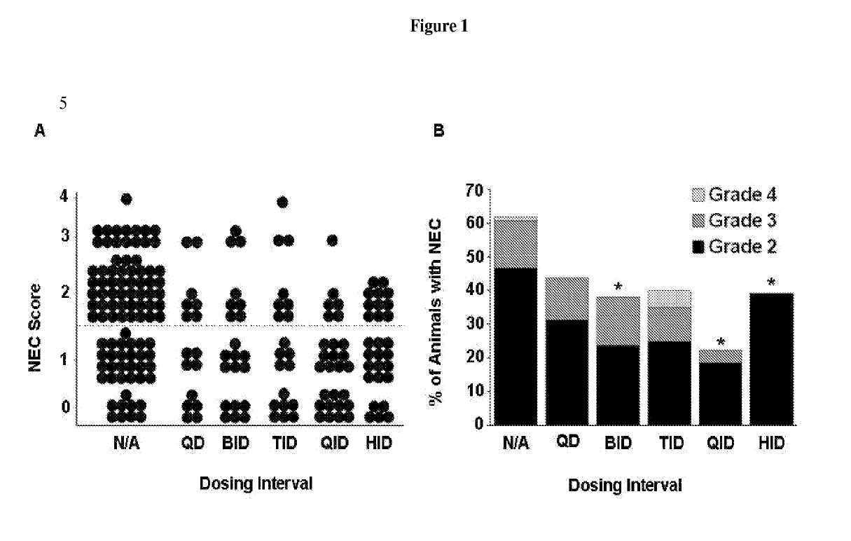

Using the neonatal rat model of NEC, as described in Example 1, 203 newborn rat pups were randomized to receive HB-EGF added to their feeds two (BID), three (TID), four (QID) or six (HID) equally spaced times a day. Animals subjected to stress had a 63% incidence of NEC, with histopathologic changes in the intestines ranging from moderate, mid-level villous necrosis (grade 2) to severe necrosis of the entire villous (grade 3 and grade 4) (FIGS. 1A, B). Rat pups that received HB-EGF (800 μg/kg/dose) added to every feed (6 times a day) showed a significant decrease in the incidence of NEC to 39% (p=0.03). Decreasing the HB-EGF dosing interval to either 2 or 4 times a day also significantly reduced the percent of animals that developed NEC to 38% and 22% respectively (p=0.05 and p<0.001 respectively). In addition to decreasing the incidence of NEC, addition of HB-EGF to the feeds decreased the degree of intestinal damage in the pups that did develop NEC. In non-HB-EGF-treated pups, of the 63% of pups that developed NEC, 1.7% had grade 4 injury, 24.1% had grade 3 injury and 74.1% had grade 2 injury. On the other hand, in pups treated with HB-EGF four times a day, of the 22% that did develop NEC, only 16.6% had grade 3 injury and 83.3% had grade 2 injury.

### Example 3

### Comparison of P. pastoris-Derived and E. coli-Derived HB-EGF

To compare the efficacy of E. coli-derived and P. pastoris-derived HB-EGF, 199 rat pups were randomized to receive 600, 800 or 1000 μg/kg/dose of each type of HB-EGF added to their feeds 4 or 6 times a day using the neonatal rat model of NEC as described in Example 1. The HB-EGF used in all experiments was GMP grade human mature HB-EGF produced in P. pastoris yeast (KBI BioPharma, Inc., Durham, N.C.). E. coli-derived recombinant human mature HB-EGF produced as previously described (Davis et al., Protein Expr Purif 8:57-67, 1996) was used. Previous studies of the ability of E. coli-derived HB-EGF to prevent NEC tested doses up to but not exceeding 800 μg/kg/dose. Thus, the effect of increasing the dose of HB-EGF to 1000 μg/kg/dose was also tested. In this experiment, the incidence of NEC in stressed pups was 68%. When tested at doses of 600, 800, or 1000 μg/kg/dose, and dosing intervals of 4 or 6 times a day, there were no significant differences in efficacy between E. coli-derived and Pichia-derived HB-EGF. Increasing the dose of HB-EGF to 1000 μg/kg/dose did not result in a further beneficial effect.

### Example 4

### Comparison of HB-EGF and EGF in Prevention of NEC

To compare the efficacy of HB-EGF and EGF in the prevention of NEC, the neonatal rat model of NEC as described in Example 1 was used. One hundred and twenty rat pups were randomized to receive either equal mass doses of each growth factor (HB-EGF 800 μg/kg/dose vs. EGF 800 μg/kg/dose) or molar equivalents of each growth factor (HB-EGF 800 μg/kg/dose vs. EGF 570 μg/kg/dose).

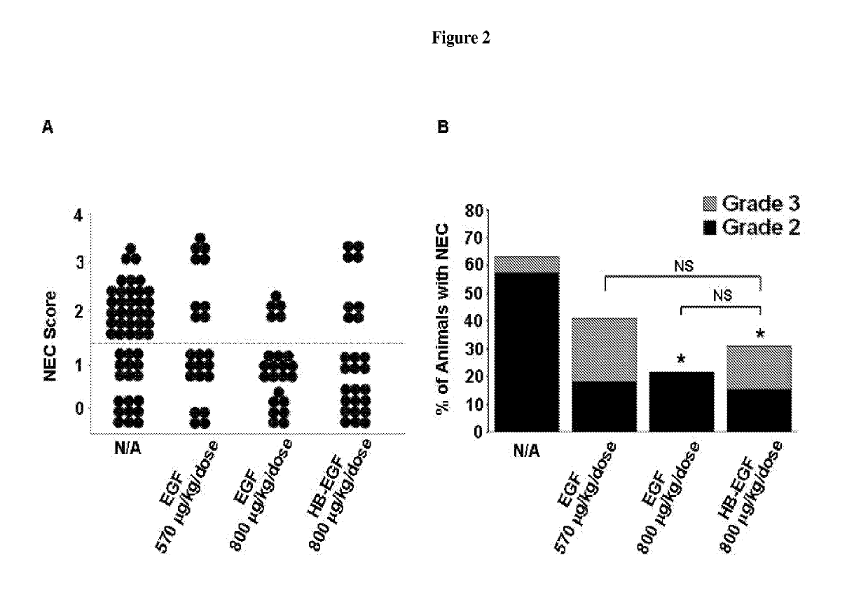

A dose of HB-EGF (800 μg/kg/dose) with proven efficacy in preventing NEC was chosen, and compared this dose to both the equivalent mass dose of EGF (800 μg/kg/dose) as well as the equivalent molar dose of EGF (570 μg/kg/dose). Comparing equal molar doses of the two growth factors takes into account the different molecular masses of the mature forms of the two growth factors used in this study (i.e., HB-EGF residues 74-148; [74aa; Mr7400] vs. EGF residues 1-53 [53aa; Mr 5300]), and adds an equal number of molecules of each growth factor to the experiment. In this experiment, animals subjected to stress had an incidence of NEC of 63.3% (FIGS. 2A, B). HB-EGF (800 μg/kg/dose) significantly decreased the incidence of NEC to 30.7% (p=0.009). The equivalent mass dose of EGF (800 μg/kg/dose) significantly decreased the incidence of NEC to 21.7% (p=0.002), and the equivalent molar dose (570 μg/kg/dose) decreased the incidence of NEC to 40.9% (p=0.12). There were no statistically significant differences in the incidence of NEC between HB-EGF and either of the two doses of EGF tested.

In a recent report, Dvorak et al. compared the effect of enteral administration of HB-EGF compared with EGF in protection from experimental NEC in newborn rats (J Ped Gastroenterol and Nutr 47:11-18, 2008). The authors concluded that both growth factors could protect rat pups from developing NEC, but suggested that EGF may be effective at more physiologic levels. The basis of that conclusion is not totally clear, since both growth factors in their study had maximal beneficial effects at the same dose (500 ng/ml). There are several difficulties encountered when trying to compare the results of the Dvorak study with those described herein. First, Dvorak et al. report their doses of growth factors administered in ng/ml rather than in ng or μg/kg/dose. The rat pups in the present study received doses measured in μg/kg/dose since this is directly comparable to the way in which pediatric patients are dosed in clinical practice, and since this allows for further determination of the human equivalent dose of HB-EGF using the following formula (FDA; Pharmacology and Toxicology. Jul. 1-27, 2005):

(HED=animal dose in mg/kg)×[animal weight in kg÷human weight in kg]^{0.33}

Furthermore, Dvorak et al. never state the volume (in ml) of the feeds that were administered, or the number of doses that were administered each day, making it impossible to definitively determine the exact amount of each growth factor administered. However, if assumed that Dvoaek et al. administered 0.1-0.4 ml/feed, and that their newborn rat pups weigh ˜0.005 kg, then they are delivering ˜10-40 μg/kg/dose of HB-EGF or EGF in their experiments, which is ˜20-fold less HB-EGF than the most efficacious dose of HB-EGF as described herein. In fact, using the NEC injury grading system used herein, which is the same system proposed by Caplan et al. (Pediatr Pathol 14:1017-28, 1994), the doses used by Dvorak et al. would not show any beneficial effect. This may be attributed to the fact that different injury scoring systems are being used in the studies of Dvork et al. and herein.

### Example 5

### Comparison of Prophylactic and Therapeutic Administration of HB-EGF in the Prevention of NEC

The invention contemplates prophylactic clinical administration of HB-EGF for NEC in an attempt to prevent NEC from developing, or therapeutically in an attempt to reverse or inhibit progression of NEC that has already occurred. Previously, a rodent model of intestinal ischemia/reperfusion injury secondary to superior mesenteric artery occlusion was used to show that HB-EGF can significantly protect the intestines from injury when administered either prophylacticly or therapeutically, however the best results were obtained when HB-EGF was administered prior to injury (Martin et al., J Pediatr Surg 40:1741-7, 2005). Similar experiments using the neonatal rodent model of NEC have not been previously performed.

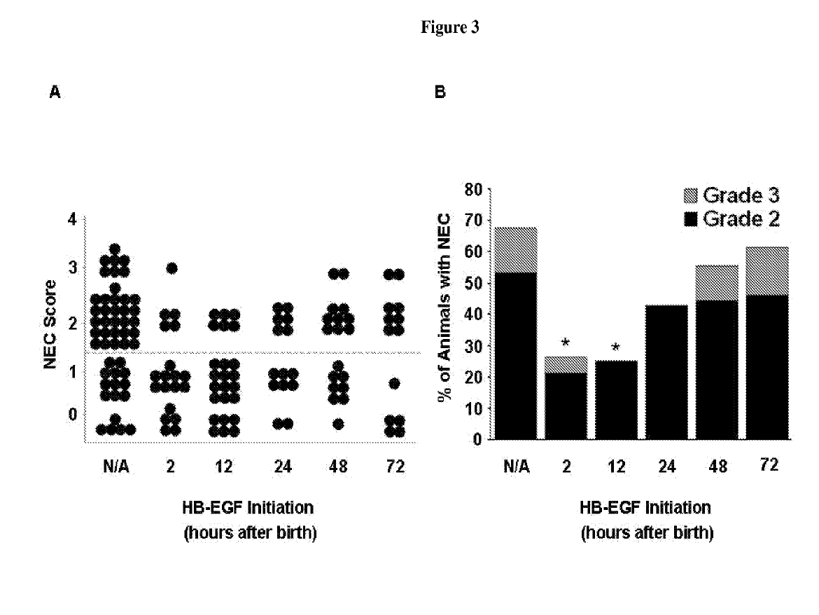

Rat pups were exposed to stress beginning immediately after birth using the model described in Example 1, with addition of HB-EGF (800 μg/kg/dose) to the feeds beginning with either the first feed at 2 h after birth (prophylactic administration), or beginning after 12, 24, 48 or 72 hours after birth. In this experiment, the incidence of NEC in stressed animals was 67.3% (FIG. 3). The incidence of NEC decreased significantly to 26.3% when HB-EGF was added to the feeds starting at 2 h, and to 25.0% when HB-EGF was started at 12 h after birth (p=0.003 and p=0.001, respectively). In addition to decreasing the incidence of NEC, HB-EGF supplementation of the formula at the 2 h or 12 h time points decreased the degree of intestinal damage in the pups that did develop NEC. Of the 67.3% of stressed animals that developed NEC, 78.8% had grade 2 injury and 21.2% had grade 3 injury. In animals that received HB-EGF starting 2 h after birth, of the 26.3% that went on to develop NEC, only 20% had grade 3 injury and 80% had grade 2 injury. In pups that received HB-EGF starting 12 h after birth, of the 25% that went on to develop NEC, none had grade 3 injury and 100% had grade 2 injury. When HB-EGF administration was started at later time points (24, 48 and 72 h), there were no significant differences in the incidence or severity of NEC compared to control animals.

### Example 6

### HB-EGF Knock Out Mice Exhibit Increased Susceptibility to NEC

The role of endogenous HB-EGF gene expression in susceptibility to intestinal injury and the preservation of gut barrier function in a newborn mouse model of experimental NEC using HB-EGF Knock Out (KO) mice was investigated. HB-EGF knock out (KO) mice on a C57BLI6J×129 background and HB-EGF WT C57BL/6J×129 mice as described by Jackson et al. (EMBO J. 22: 2704-2716, 2003) were used. In the HB-EGF KO mice, HB-EGF exons 1 and 2 were replaced with PCK-Neo, thus deleting the signal peptide and propeptide domains. The desired targeting events were verified by Southern blots of genomic DNA and exon-specific polymerase chain reaction, with Northern blots confirming the absence of the respective transcripts.

NEC was induced using the experimental model described in Example 1 as modified for mice as described by filling et al. (J. Immunol. 177: 3273-3282, 25006). Pregnant time-dated mice were delivered by C section under inhaled 2% Isofturane (Butler Animal Health, Dublin, Ohio) anesthesia on day 18.5 of gestation. Newborn mouse pups were placed in an incubator (37° C.) and fed via gastric gavage with formula containing 15 g Similac 60/40 (Ross Pediatrics, Columbus, Ohio) in 75 mL Esbilac (Pet-Ag, New Hampshire, Ill.), providing 836.8 kJ/kg per day. Feeds were started at 0.03 mL every 3 hours beginning 2 hours after birth and advanced as tolerated up to a maximum of 0.05 mL per feeding by the fourth day of life. Animals were stressed by exposure to hypoxia (100% nitrogen for 1 minute) followed by hypothermia (4° C. for 10 minutes) once a day beginning immediately after birth until the end of the experiment. Exposure of pups to hypoxia, hypothermia and hypertonic feeds will subsequently be referred to herein as exposure to “stress”.

To investigate the effects of HB-EGF loss-of-function on susceptibility to NEC, HB-EGF WT pups (n=19) and HB-EGF KO pups (n=31) were exposed to experimental NEC. An additional group of HB-EGF KO pups (n=33) were exposed to experimental NEC as described, but received HB-EGF (800 pg/kg/dose) added to each feed (starting 2 hours after birth). The HB-EGF used was Good Manufacturing Practice (GMP) grade human mature HB-EGF produced in Pichia pastoris yeast (Trillium Therapeutics, Inc., Toronto, Canada). In all experiments, pups were euthanized upon development of clinical signs of NEC (abdominal distention, bloody bowel movements, respiratory distress, and lethargy). Remaining animals were sacrificed 96 hours after birth.

### Histologic Injury

Upon sacrifice, the gastrointestinal tract was carefully removed and visually evaluated for signs of NEC (areas of bowel necrosis, intestinal hemorrhage, perforation). Three pieces of duodenum, jejunum, ileum, and colon from every animal were fixed in 10% formalin for 24 hours, paraffin-embedded, sectioned at 5 μm thickness, and stained with hematoxylin and eosin for histological evaluation of the presence and/or degree of NEC using the NEC histologic injury scoring system described by Caplan et al. (Pediatric Pathol. 14: 1017-1028, 2007) Histological changes were graded as follows: grade 0: no damage; grade 1: epithelial cell lifting or separation; grade 2: sloughing of epithelial cells to the mid villus level; grade 3: necrosis of the entire villus; and grade 4: transmural necrosis. Tissues were graded blindly by two independent observers. Tissues with histological scores of 2 or higher were considered positive for NEC.

Histologic analyses revealed that HB-EGF WT mouse pups had an incidence of NEC of 53%, with grade 2 injury seen in 100% of the animals that developed NEC. HB-EGF KO mice had a significantly increased incidence of NEC of 80% (p=0.04), with histopathologic changes ranging from moderate, mid-level villous necrosis (grade 2) to severe necrosis of the entire villous (grade 3). Of the 80% of pups that developed NEC, 48% had grade 2 injury and 32% had grade 3 injury. HB-EGF KO pups exposed to stress but with HB-EGF (800 μg/kg/dose) added to the feeds showed a significant decrease in the incidence of NEC to 45% compared to stressed pups that were not treated with HB-EGF (p=0.004). In addition to a decreased incidence of NEC, supplementation of HB-EGF to the formula of HB-EGF KO pups resulted in decreased severity of NEC. Of the 45% of HB-EGF-treated pups that developed NEC, 44% had grade 2 injury and only 3% had grade 3 injury.

### Gut Barrier Function

Intestinal permeability was also examined to determine gut barrier function in HB-EGF WT and HB-EGF KO mice exposed to experimental NEC. Fluorescein isothiocyanate (FITC)-labeled dextran molecules (molecular weight, 73 kDa) (Sigma-Aldrich Inc, St Louis, Mo.) was used as a probe to examine gut barrier function. Previous studies by others have shown that use of 73-kDa dextran molecules results in a reliable assessment of mucosal perturbations 4 hours after enteral administration (Caplan et al. Gastroenterology 117:577-583, 1999). In this experiment, FITC-labeled dextran molecules (750 mg/kg) were administered via orogastric tube to mouse pups. After 4 hours, blood was collected and plasma FITC-dextran levels were measured using spectrophotofluorometry (Molecular Devices, SpectraMax M2, Sunnyvale, Ca). The amount of dextran in the plasma was calculated based on standard dilution curves of known dextran concentrations. The mouse pups were divided into 4 groups as follows: 1) WT mice that received intragastric FITC-dextran immediately after birth with no exposure to stress (n=15); 2) HB-EGF KO mice that received intragastric FITC-dextran immediately after birth with no exposure to stress (n=17); 3) HB-EGF WT mice that received intragastric FITC dextran after 24 hours of stress (n=13); and 4) HB-EGF KO mice that received intragastric FITC dextran after 24 hours of stress (n=10).

The Chi-square test was used for comparing the incidence of NEC between groups. Serum concentrations of FITC-dextran were compared using the Student's t test. p-values less then 0.05 were considered statistically significant. All statistical analyses were performed using SAS software (Version 9.1, SAS Institute, Cary, N.C.).

Under basal, non-stressed conditions immediately after birth, HB-EGF KO pups had significantly increased serum FITC-dextran levels compared to HB-EGF WT pups (179.73±58.43 μg/ml vs. 47.79±14.39 μg/ml; p=0.04). After 24 hours of exposure to stress, HB-EGF WT mice had increased serum FITC-dextran levels compared to HB-EGF WT mice under basal conditions (119.86±36.39 μg/ml vs. 47.79±14.39 μ/ml; p=0.00003). On the other hand, HB-EGF KO pups exposed to stress for 24 hours had a much smaller increase in serum FITC-dextran levels compared to KO mice under basal conditions (190.70±61.54 μg/ml vs. 179.73±58.43 μg/ml), but still had much higher serum FITC-dextran levels compared to WT mice exposed to stress for 24 hours (190.70±61.54 μg/ml vs. 119.86±36.39 μg/ml; p=0.3). The FITC-dextran serum levels in WT animals after birth are low, indicating intact intestinal barrier function, but as the animals are exposed to stress for 24 hours there is an increase in serum FITC-dextran levels indicating damage to the mucosal barrier. HB-EGF KO mice have increased FITC-dextran serum levels immediately after birth and maintain high serum levels at the 24 hour time point as well, suggesting a baseline deficit in gut barrier function that may explain, in part, their increased susceptibility to NEC.

These experiments demonstrate that newborn HB-EGF KO mice have increased susceptibility to experimental NEC, and show that they have increased intestinal permeability under both basal and stressed conditions. The effects of lack of endogenous HB-EGF on the intestine can be compensated for by administration of exogenous enteral HB-EGF. These findings support the concept of administration of HB-EGF to patients with or at risk of developing NEC in order to prevent the progression of or development of the disease.

Studies in critically ill adults have shown that impairment of mucosal barrier function with overgrowth of pathogenic bacteria in the gastrointestinal tract enhances translocation of bacteria and endotoxin, resulting in a septic inflammatory response and multiorgan failure (Deitch, Arch Surg 125:403-404, 1990; Hadfield et al. Am. J. Respir. Crit. Care Med. 152:1545-1548, 1995). Plena-Spoel et al. (J. Pediat. Surg. 36: 587-592, 2001) evaluated changes in intestinal permeability in 13 children with NEC compared to 10 control patients undergoing surgery by measuring lactulose to rhamnose ratios in urine samples. They found that lactulose to rhamnose ratios in NEC patients were increased for prolonged periods of time, with high peaks seen in patients with sepsis, indicative of gut barrier failure. Control patients had increased intestinal permeability only in the first days after surgery, which normalized rapidly afterwards. Beach et al. (Arch. Dis. Childhood, 57: 141-145, 1982) observed increased intestinal permeability during the first week of life in neonates of gestational age 31-36 weeks, while Weaver (Arch. Dis. Childhood, 59: 236-241, 1984) showed that premature newborns born prior to 34 weeks gestation exhibited higher intestinal permeability than more mature newborns. The impaired gut barrier function of premature babies under basal conditions may be similar to the impaired intestinal permeability reported here in newborn HB-EGF KO mice under basal conditions. When HB-EGF expression is decreased or absent, as in the intestine of neonates afflicted with NEC or in HB-EGF KO mice, gut barrier function is impaired, which may contribute to bacterial translocation leading to a systemic inflammatory response.

The results of the current study, demonstrating increased intestinal injury and increased intestinal permeability in HB-EGF KO mice exposed to experimental NEC, support the contention that HB-EGF expression is important in protection of the intestines from NEC. The fact that administration of exogenous HB-EGF to HB-EGF KO mice protects the intestines from experimental NEC supports the clinical administration of HB-EGF to patients with or at risk of developing NEC in an effort to treat or prevent the disease.

1. A method of treating an infant suffering from or at risk for necrotizing enterocolitis (NEC), comprising administering an EGF receptor agonist in an amount effective to reduce the onset or severity of NEC, wherein the EGF receptor agonist is administered within 24 hours following birth.

2. A method of treating an infant to abate necrotizing enterocolitis (NEC), comprising administering an amount of an EGF receptor agonist in an amount effective to reduce the onset of NEC or severity of NEC, wherein the EGF receptor agonist is administered within 24 hours following birth.

3. A method of reducing the risk of developing necrotizing enterocolitis (NEC) in an infant, comprising administering an EGF receptor agonist in an amount effective to reduce the onset of NEC, wherein the EGF receptor agonist is administered within 24 hours following birth.

4. A method of treating an infant suffering from necrotizing enterocolitis (NEC), comprising administering an EGF receptor agonist in an amount effective to reduce the onset or severity of NEC, wherein the EGF receptor agonist is administered within 24 hours following onset of at least one symptom of NEC.

5. The method of claim 1, wherein the EGF receptor agonist is a HB-EGF product.

6. The method of claim 5, wherein the HB-EGF product comprises amino acids of 74-148 of SEQ ID NO: 2.

7. The method of claim 1, wherein the EGF receptor agonist is an EGF product.

8. The method of claim 7, wherein the EGF product comprises amino acids 1-53 of SEQ ID NO: 4.

9. The method of claim 1, wherein the effective amount of EGF receptor agonist is 100-140 μg/kg dose.

10. The method of claim 1, wherein the effective amount of EGF receptor agonist is 100 μg/kg dose.

11-14. (canceled)

15. The method of claim 9, wherein the dose is administered twice a day, four times a day, six times a day or eight times a day.

16. (canceled)

17. The method of claim 1, wherein the dose is administered within 2 hours following birth.

18. The method of claim 1, wherein the dose is administered within 12 hours following birth.

19. The method of claim 4, wherein the dose is administered within 2 hours following the onset of at least one symptom of NEC.

20. The method of claim 4, wherein the dose is administered within 12 hours following the onset of at least one symptom of NEC.

21. The method of claim 1 wherein the infant is a premature infant.


SEQUENCE LISTING (16 sequences)

SEQ ID NO: 1 - DNA, 624 bp, Homo sapiens
  CDS (1)..(624)
atgaagctgctgccgtcggtggtgctgaagctctttctggctgcagttctctcggcactggtgactggcgagagcctggagcggcttcggagagggctagctgctggaaccagcaacccggaccctcccactgtatccacggaccagctgctacccctaggaggcggccgggaccggaaagtccgtgacttgcaagaggcagatctggaccttttgagagtcactttatcctccaagccacaagcactggccacaccaaacaaggaggagcacgggaaaagaaagaagaaaggcaaggggctagggaagaagagggacccatgtcttcggaaatacaaggacttctgcatccatggagaatgcaaatatgtgaaggagctccgggctccctcctgcatctgccacccgggttaccatggagagaggtgtcatgggctgagcctcccagtggaaaatcgcttatatacctatgaccacacaaccatcctggccgtggtggctgtggtgctgtcatctgtctgtctgctggtcatcgtggggcttctcatgtttaggtaccataggagaggaggttatgatgtggaaaatgaagagaaagtgaagttgggcatgactaattcccac

SEQ ID NO: 2 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 3 - DNA, 4913 bp, Homo sapiens
aaaaagagaaactgttgggagaggaatcgtatctccatatttcttctttcagccccaatccaagggttgtagctggaactttccatcagttcttcctttctttttcctctctaagcctttgccttgctctgtcacagtgaagtcagccagagcagggctgttaaactctgtgaaatttgtcataagggtgtcaggtatttcttactggcttccaaagaaacatagataaagaaatctttcctgtggcttcccttggcaggctgcattcagaaggtctctcagttgaagaaagagcttggaggacaacagcacaacaggagagtaaaagatgccccagggctgaggcctccgctcaggcagccgcatctggggtcaatcatactcaccttgcccgggccatgctccagcaaaatcaagctgttttcttttgaaagttcaaactcatcaagattatgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaatgagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggatggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgatattgatgagtgccaactgggggagcacagctgtggagagaatgccagctgcacaaatacagagggaggctatacctgcatgtgtgctggacgcctgtctgaaccaggactgatttgccctgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtgaaaactggaattaaaaggaaagtcaagaagaatgaactatgtcgatgcacagtatcttttctttcaaaagtagagcaaaactataggttttggttccacaatctctacgactaatcacctactcaatgcctggagacagatacgtagttgtgcttttgtttgctcttttaagcagtctcactgcagtcttatttccaagtaagagtactgggagaatcactaggtaacttattagaaacccaaattgggacaacagtgctttgtaaattgtgttgtcttcagcagtcaatacaaatagatttttgtttttgttgttcctgcagccccagaagaaattaggggttaaagcagacagtcacactggtttggtcagttacaaagtaatttctttgatctggacagaacatttatatcagtttcatgaaatgattggaatattacaataccgttaagatacagtgtaggcatttaactcctcattggcgtggtccatgctgatgattttgcaaaatgagttgtgatgaatcaatgaaaaatgtaatttagaaactgatttcttcagaattagatggcttattttttaaaatatttgaatgaaaacattttatttttaaaatattacacaggaggcttcggagtttcttagtcattactgtccttttcccctacagaattttccctcttggtgtgattgcacagaatttgtatgtattttcagttacaagattgtaagtaaattgcctgatttgttttcattatagacaacgatgaatttcttctaattatttaaataaaatcaccaaaaacataaaaaaaaaaaaaaaaaaaaaaaaaaaaa

SEQ ID NO: 4 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 5 - DNA, 4261 bp, Homo sapiens
agccgccttcctatttccgcccggcgggcagcgctgcggggcgagtgccagcagagaggcgctcggtcctccctccgccctcccgcgccgggggcaggccctgcctagtctgcgtctttttcccccgcaccgcggcgccgctccgccactcgggcaccgcaggtagggcaggaggctggagagcctgctgcccgcccgcccgtaaaatggtcccctcggctggacagctcgccctgttcgctctgggtattgtgttggctgcgtgccaggccttggagaacagcacgtccccgctgagtgacccgcccgtggctgcagcagtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccgtggtggctgccagccagaagaagcaggccatcaccgccttggtggtggtctccatcgtggccctggctgtccttatcatcacatgtgtgctgatacactgctgccaggtccgaaaacactgtgagtggtgccgggccctcatctgccggcacgagaagcccagcgccctcctgaagggaagaaccgcttgctgccactcagaaacagtggtctgaagagcccagaggaggagtttggccaggtggactgtggcagatcaataaagaaaggcttcttcaggacagcactgccagagatgcctgggtgtgccacagaccttcctacttggcctgtaatcacctgtgcagccttttgtgggccttcaaaactctgtcaagaactccgtctgcttggggttattcagtgtgacctagagaagaaatcagcggaccacgatttcaagacttgttaaaaaagaactgcaaagagacggactcctgttcacctaggtgaggtgtgtgcagcagttggtgtctgagtccacatgtgtgcagttgtcttctgccagccatggattccaggctatatatttctttttaatgggccacctccccacaacagaattctgcccaacacaggagatttctatagttattgttttctgtcatttgcctactggggaagaaagtgaaggaggggaaactgtttaatatcacatgaagaccctagctttaagagaagctgtatcctctaaccacgagaccctcaaccagcccaacatcttccatggacacatgacattgaagaccatcccaagctatcgccacccttggagatgatgtcttatttattagatggataatggttttatttttaatctcttaagtcaatgtaaaaagtataaaaccccttcagacttctacattaatgatgtatgtgttgctgactgaaaagctatactgattagaaatgtctggcctcttcaagacagctaaggcttgggaaaagtcttccagggtgcggagatggaaccagaggctgggttactggtaggaataaaggtaggggttcagaaatggtgccattgaagccacaaagccggtaaatgcctcaatacgttctgggagaaaacttagcaaatccatcagcagggatctgtcccctctgttggggagagaggaagagtgtgtgtgtctacacaggataaacccaatacatattgtactgctcagtgattaaatgggttcacttcctcgtgagccctcggtaagtatgtttagaaatagaacattagccacgagccataggcatttcaggccaaatccatgaaagggggaccagtcatttattttccattttgttgcttggttggtttgttgctttatttttaaaaggagaagtttaactttgctatttattttcgagcactaggaaaactattccagtaatttttttttcctcatttccattcaggatgccggctttattaacaaaaactctaacaagtcacctccactatgtgggtcttcctttcccctcaagagaaggagcaattgttcccctgagcatctgggtccatctgacccatggggcctgcctgtgagaaacagtgggtcccttcaaatacatagtggatagctcatccctaggaattttcattaaaatttggaaacagagtaatgaagaaataatatataaactccttatgtgaggaaatgctactaatatctgaaaagtgaaagatttctatgtattaactcttaagtgcacctagcttattacatcgtgaaaggtacatttaaaatatgttaaattggcttgaaattttcagagaattttgtcttcccctaattcttcttccttggtctggaagaacaatttctatgaattttctctttatttttttttataattcagacaattctatgacccgtgtcttcatttttggcactcttatttaacaatgccacacctgaagcacttggatctgttcagagctgaccccctagcaacgtagttgacacagctccaggtttttaaattactaaaataagttcaagtttacatcccttgggccagatatgtgggttgaggcttgactgtagcatcctgcttagagaccaatcaacggacactggtttttagacctctatcaatcagtagttagcatccaagagactttgcagaggcgtaggaatgaggctggacagatggcggaagcagaggttccctgcgaagacttgagatttagtgtctgtgaatgttctagttcctaggtccagcaagtcacacctgccagtgccctcatccttatgcctgtaacacacatgcagtgagaggcctcacatatacgcctccctagaagtgccttccaagtcagtcctttggaaaccagcaggtctgaaaaagaggctgcatcaatgcaagcctggttggaccattgtccatgcctcaggatagaacagcctggcttatttggggatttttcttctagaaatcaaatgactgataagcattggatccctctgccatttaatggcaatggtagtctttggttagctgcaaaaatactccatttcaagttaaaaatgcatcttctaatccatctctgcaagctccctgtgtttccttgccctttagaaaatgaattgttcactacaattagagaatcatttaacatcctgacctggtaagctgccacacacctggcagtggggagcatcgctgtttccaatggctcaggagacaatgaaaagcccccatttaaaaaaataacaaacattttttaaaaggcctccaatactcttatggagcctggatttttcccactgctctacaggctgtgactttttttaagcatcctgacaggaaatgttttcttctacatggaaagatagacagcagccaaccctgatctggaagacagggccccggctggacacacgtggaaccaagccagggatgggctggccattgtgtccccgcaggagagatgggcagaatggccctagagttcttttccctgagaaaggagaaaaagatgggattgccactcacccacccacactggtaagggaggagaatttgtgcttctggagcttctcaagggattgtgttttgcaggtacagaaaactgcctgttatcttcaagccaggttttcgagggcacatgggtcaccagttgctttttcagtcaatttggccgggatggactaatgaggctctaacactgctcaggagacccctgccctctagttggttctgggctttgatctcttccaacctgcccagtcacagaaggaggaatgactcaaatgcccaaaaccaagaacacattgcagaagtaagacaaacatgtatatttttaaatgttctaacataagacctgttctctctagccattgatttaccaggctttctgaaagatctagtggttcacacagagagagagagagtactgaaaaagcaactcctcttcttagtcttaataatttactaaaatggtcaacttttcattatctttattataataaacctgatgcttttttttagaactccttactctgatgtctgtatatgttgcactgaaaaggttaatatttaatgttttaatttattttgtgtggtaagttaattttgatttctgtaatgtgttaatgtgattagcagttattttccttaatatctgaattatacttaaagagtagtgagcaatataagacgcaattgtgtttttcagtaatgtgcattgttattgagttgtactgtaccttatttggaaggatgaaggaatgaatctttttttcctaaa

SEQ ID NO: 6 - protein, 159 aa, Homo sapiens
MVPSAGQLALFALGIVLAACQALENSTSPLSDPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 7 - DNA, 1270 bp, Homo sapiens
agacgttcgcacacctgggtgccagcgccccagaggtcccgggacagcccgaggcgccgcgcccgccgccccgagctccccaagccttcgagagcggcgcacactcccggtctccactcgctcttccaacacccgctcgttttggcggcagctcgtgtcccagagaccgagttgccccagagaccgagacgccgccgctgcgaaggaccaatgagagccccgctgctaccgccggcgccggtggtgctgtcgctcttgatactcggctcaggccattatgctgctggattggacctcaatgacacctactctgggaagcgtgaaccattttctggggaccacagtgctgatggatttgaggttacctcaagaagtgagatgtcttcagggagtgagatttcccctgtgagtgaaatgccttctagtagtgaaccgtcctcgggagccgactatgactactcagaagagtatgataacgaaccacaaatacctggctatattgtcgatgattcagtcagagttgaacaggtagttaagcccccccaaaacaagacggaaagtgaaaatacttcagataaacccaaaagaaagaaaaagggaggcaaaaatggaaaaaatagaagaaacagaaagaagaaaaatccatgtaatgcagaatttcaaaatttctgcattcacggagaatgcaaatatatagagcacctggaagcagtaacatgcaaatgtcagcaagaatatttcggtgaacggtgtggggaaaagtccatgaaaactcacagcatgattgacagtagtttatcaaaaattgcattagcagccatagctgcctttatgtctgctgtgatcctcacagctgttgctgttattacagtccagcttagaagacaatacgtcaggaaatatgaaggagaagctgaggaacgaaagaaacttcgacaagagaatggaaatgtacatgctatagcataactgaagataaaattacaggatatcacattggagtcactgccaagtcatagccataaatgatgagtcggtcctctttccagtggatcataagacaatggaccctttttgttatgatggttttaaactttcaattgtcactttttatgctatttctgtatataaaggtgcacgaaggtaaaaagtattttttcaagttgtaaataatttatttaatatttaatggaagtgtatttattttacagctcattaaacttttttaaccaaacagaaaaaaaaaaaaaaaaaaaaaaaaaaaaaaaaa

SEQ ID NO: 8 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 9 - DNA, 1323 bp, Homo sapiens
gcccgaatatgtccctgggtgtgggtatgggtgtggggcaatttgggtgggagcagcgtggaggctcccaggaccaagtcctgcgcctctttggcggggtgtgtgcaggaggaggggggataaataggaggctccctcctcccggcgacattcacggagccggccggcctcccgccctgggtgtttccctgccttgtagccagggtgccagcctgggaagtagtttcgtttccttctgcctccgggattagtttccaggcaccctctcaggcgcccgaggcccgggaagggggcgaagaaggagggagacttgtctaggggctgcccggcccggcagagcggggttgatggaccgggccgcccggtgcagcggcgccagctccctgccactgctcctggcccttgccctgggtctagtgatccttcactgtgtggtggcagatgggaattccaccagaagtcctgaaactaatggcctcctctgtggagaccctgaggaaaactgtgcagctaccaccacacaatcaaagcggaaaggccacttctctaggtgccccaagcaatacaagcattactgcatcaaagggagatgccgcttcgtggtggccgagcagacgccctcctgtgtctgtgatgaaggctacattggagcaaggtgtgagagagttgacttgttttacctaagaggagacagaggacagattctggtgatttgtttgatagcagttatggtagtttttattattttggtcatcggtgtctgcacatgctgtcaccctcttcggaaacgtcgtaaaagaaagaagaaagaagaagaaatggaaactctgggtaaagatataactcctatcaatgaagatattgaagagacaaatattgcttaaaaggctatgaagttacctccaggttggtggcaagctgcaaagtgccttgctcatttgaaaatggacagaatgtgtctcaggaaaacagctagtagacatgaattttaaataatgtatttactttttatttgcaactttagtttgtgttattattttttaataagaacattaattatatgtatattgtctagtaattgggaaaaaagcaactggttaggtagcaacaacagaagggaaatttcaataacctttcacttaagtattgtcaccaggattactagtcaaacaaaaaagaaaagtagaaaggaggttaggtcttaggaattgaattaataataaagctaccatttatcaagcatttaccatgtgctaataagtttgaaatatattatttcctttattcctttcagcaatccatgagatagctattataatcctc

SEQ ID NO: 10 - protein, 178 aa, Homo sapiens
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICLIAVMVVFIILVIGVCTCCHPLRKRRKRKKKEEEMETLGKDITPINEDIEETNIA

SEQ ID NO: 11 - DNA, 4628 bp, Homo sapiens
tcacttgcctgatatttccagtgtcagagggacacagccaacgtggggtcccttctaggctgacagccgctctccagccactgccgcgagcccgtctgctcccgccctgcccgtgcactctccgcagccgccctccgccaagccccagcgcccgctcccatcgccgatgaccgcggggaggaggatggagatgctctgtgccggcagggtccctgcgctgctgctctgcctgggtttccatcttctacaggcagtcctcagtacaactgtgattccatcatgtatcccaggagagtccagtgataactgcacagctttagttcagacagaagacaatccacgtgtggctcaagtgtcaataacaaagtgtagctctgacatgaatggctattgtttgcatggacagtgcatctatctggtggacatgagtcaaaactactgcaggtgtgaagtgggttatactggtgtccgatgtgaacacttctttttaaccgtccaccaacctttaagcaaagaatatgtggctttgaccgtgattcttattattttgtttcttatcacagtcgtcggttccacatattatttctgcagatggtacagaaatcgaaaaagtaaagaaccaaagaaggaatatgagagagttacctcaggggatccagagttgccgcaagtctgaatggcgccatcaaacttatgggcagggataacagtgtgcctggttaatattaatattcccattttattaataatatttatgttgggtcaagtgttaggtcaataacactgtattttaatgtacttgaaaaatgtttttatttttgttttatttttgacagactatttgctaatgtataatgtgcagaaaatatttaatatcaaaagaaaattgatatttttatacaagtaatttcctgagctaaatgcttcattgaaagcttcaaagtttatatgcctggtgcacagtgcttagaagtaagcaattcccaggtcatagctcaagaattgttagcaaatgacagatttctgtaagcctatatatatagtcaaatcgatttagtaagtatgttttttatgttcctcaaatcagtgataattggtttgactgtaccatggtttgatatgtagttggcaccatggtatcatatattaaaacaataatgcaattagaatttgggagaagcaaatataggtcctgtgttaaacactacacatttgaaacaagctaaccctggggagtctatggtctcttcactcaggtctcagctataattctgttatatgaggggcagtggacagttccctatgccaactcacgactcctacaggtactagtcactcatctaccagattctgcctatgtaaaatgaattgaaaaacaattttctgtaatcttttatttaagtagtgggcatttcatagcttcacaatgttccttttttgtatattacaacatttatgtgaggtaattattgctcaacagacaattagaaaaaagtccacacttgaagcctaaatttgtgctttttaagaatatttttagactatttctttttataggggctttgctgaattctaacattaaatcacagcccaaaatttgatggactaattattattttaaaatatatgaagacaataattctacatgttgtcttaagatggaaatacagttatttcatcttttattcaaggaagttttaactttaatacagctcagtaaatggcttcttctagaatgtaaagttatgtatttaaagttgtatcttgacacaggaaatgggaaaaaacttaaaaattaatatggtgtatttttccaaatgaaaaatctcaattgaaagcttttaaaatgtagaaacttaaacacaccttcctgtggaggctgagatgaaaactagggctcattttcctgacatttgtttattttttggaagagacaaagatttcttctgcactctgagcccataggtctcagagagttaataggagtatttttgggctattgcataaggagccactgctgccaccacttttggattttatgggaggctccttcatcgaatgctaaacctttgagtagagtctccctggatcacataccaggtcagggaggatctgttcttcctctacgtttatcctggcatgtgctagggtaaacgaaggcataataagccatggctgacctctggagcaccaggtgccaggacttgtctccatgtgtatccatgcattatataccctggtgcaatcacacgactgtcatctaaagtcctggccctggcccttactattaggaaaataaacagacaaaaacaagtaaatatatatggtcatatacatattgtatatatattcatatacaaacatgtatgtatacatgaccttaatggatcatagaattgcagtcatttggtgctctgctaaccatttatataaaacttaaaaacaagagaaaagaaaaatcaattagatctaaacagttatttctgtttcctatttaatacagctgaagtcaaaatatgtaagaacacattttaaatactctacttacagttggccctctgtggttagttccacatctgtggattcaaccaaccaaggacggaaaatgcttaaaaaataatacaacaacaacaaaaaatacattataacaactatttacttttttttttttctttttgagatggagtctcgctctgttgcccaggttggagtgcagtggcacgatctcggctcactgcaacctcacctcccgggttcaagagatcctcctgcctcagcctcctgagcagctgggactacaggcgcatgccaccatgcccagctaatttttgtatttttagtagaggcggggtttcaccatgttggccaggatggtctcaatctcctaaccttgagatccaccctccacagcctcccaaactgctgggattacaggtgtgagccaccgcacgtagcatttacattaggtattacaagtaatgtaaagatgatttaagtatacaggaggatgtgaataggttatatgcaagcactatgcccttttatataagtgacttgaacatctgtgcccgattttagtatgtgcaggggggcgatctgggaatcagtcccctgtggataccaaggtacaactgtatttattaacgcttactagatgtgaggagagtctgaatattttcagtgatcttggctgtttcaaaaaaatctattgacttttcaataaatcagctgcaatccatttatttcatttacaaaagatttattgtaagcatctcaatcttggtttgtcagtttatcttaagcatgtcaattcataaaaacaagtcatttttgtatttttcatctttaagaatgcttaaaaaagctaatccctaaaatagttagatctttgtaaatgcatattaaataataaagtatgacccacattactttttatgggtgaaaataagacaaaaataatagttttagtgaggatggtgctgagtaaacataaaaactgatttgctctcagctgatgtgtcctgtacacagtgggaagattttagttcacacttagtctaactcccccattttacagatttctcactatatatatttctagaaggggctatgcatattcaatgtattgagaaccaaagcaaccacaaatgcataaatgcataatttatggtcttcaaccaaggccacataataacccagttaacttactctttaaccaggaatattaagttctataactagtactcaaggtttaaccttaaaattaagatttccttaaccttaaccttaaaattgatattatattaaacatacataatacaatgtaactccactgttctcctgaatattttttgctctaatctctctgccgaaagtcaaagtgatgggagaattggtatactggtatgactacgtcttaagtcagatttttatttatgagtctttgagactaaattcaatcaccaccaggtatcaaatcaacttttatgcagcaaatatatgattctagtgtctgacttttgttaaattcagtaatgcagtttttaaaaacctgtatctgacccactttgtaatttttgctccaatatccattctgtagacttttgaaaaaaaagtttttaatttgatgcccaatatattctgaccgttaaaaaattcttgttcatatgggagaagggggagtaatgacttgtacaaacagtatttctggtgtatattttaatgtttttaaaaagagtaatttcatttaaatatctgttattcaaatttgatgatgttaaatgtaatataatgtattttctttttattttgcactctgtaattgcactttttaagtttgaagagccattttggtaaacggtttttattaaagatgctatggaacataaagttgtattgcatgcaatttgaagtaacttatttgactatgaatgttatcggattactgaattgtatcaatttgtttgtgttcaatatcagctttgataattgtgtaccttaagatattgaaggagaaaatagataatttacaagatattattaatttttatttatttttcttgggaattgaaaaaaattgaaataaataaaaatgcattgaacatcttgcattcaaaatcttcactgac

SEQ ID NO: 12 - protein, 169 aa, Homo sapiens
MTAGRRMEMLCAGRVPALLLCLGFHLLQAVLSTTVIPSCIPGESSDNCTALVQTEDNPRVAQVSITKCSSDMNGYCLHGQCIYLVDMSQNYCRCEVGYTGVRCEHFFLTVHQPLSKEYVALTVILIILFLITVVGSTYYFCRWYRNRKSKEPKKEYERVTSGDPELPQV

SEQ ID NO: 13 - DNA, 847 bp, Homo sapiens
cgtcagtctagaaggataagagaaagaaagttaagcaactacaggaaatggctttgggagttccaatatcagtctatcttttattcaacgcaatgacagcactgaccgaagaggcagccgtgactgtaacacctccaatcacagcccagcaagctgacaacatagaaggacccatagccttgaagttctcacacctttgcctggaagatcataacagttactgcatcaacggtgcttgtgcattccaccatgagctagagaaagccatctgcaggtgttttactggttatactggagaaaggtgtgagcacttgactttaacttcatatgctgtggattcttatgaaaaatacattgcaattgggattggtgttggattactattaagtggttttcttgttattttttactgctatataagaaagaggtatgaaaaagacaaaatatgaagtcacttcatatgcaatcgtttgacaaatagttattcaggccctataatgtgtcaggcactgacatgtaaaatttttttaattaaaaaagagctgtaatctggcaaaaagtttctatgtaatatttttcatgccttttctcataaacccagacgagtggtaaaaatttgccttcagttgtaataggagagttcaaacgtacagtctcccttcaacctatctctgtctgcccatatcaaaattataaatgaggaggacagcaggccccaagaaagtagggactaagtatgtcttgttcaaaattgtatattcagtgacttacactatgcctagcacacaacacacactgagtaaatatttgttgagtgaaataaaatcaagaaacaagtaaaaactga

SEQ ID NO: 14 - protein, 133 aa, Homo sapiens
MALGVPISVYLLFNAMTALTEEAAVTVTPPITAQQADNIEGPIALKFSHLCLEDHNSYCINGACAFHHELEKAICRCFTGYTGERCEHLTLTSYAVDSYEKYIAIGIGVGLLLSGFLVIFYCYIRKRYEKDKI

SEQ ID NO: 15 - DNA, 5616 bp, Homo sapiens
ccccggcgcagcgcggccgcagcagcctccgccccccgcacggtgtgagcgcccgacgcggccgaggcggccggagtcccgagctagccccggcggccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatgaccacggaggatagtatgagccctaaaaatccagactctttcgatacccaggaccaagccacagcaggtcctccatcccaacagccatgcccgcattagctcttagacccacagactggttttgcaacgtttacaccgactagccaggaagtacttccacctcgggcacattttgggaagttgcattcctttgtcttcaaactgtgaagcatttacagaaacgcatccagcaagaatattgtccctttgagcagaaatttatctttcaaagaggtatatttgaaaaaaaaaaaaagtatatgtgaggatttttattgattggggatcttggagtttttcattgtcgctattgatttttacttcaatgggctcttccaacaaggaagaagcttgctggtagcacttgctaccctgagttcatccaggcccaactgtgagcaaggagcacaagccacaagtcttccagaggatgcttgattccagtggttctgcttcaaggcttccactgcaaaacactaaagatccaagaaggccttcatggccccagcaggccggatcggtactgtatcaagtcatggcaggtacagtaggataagccactctgtcccttcctgggcaaagaagaaacggaggggatggaattcttccttagacttacttttgtaaaaatgtccccacggtacttactccccactgatggaccagtggtttccagtcatgagcgttagactgacttgtttgtcttccattccattgttttgaaactcagtatgctgcccctgtcttgctgtcatgaaatcagcaagagaggatgacacatcaaataataactcggattccagcccacattggattcatcagcatttggaccaatagcccacagctgagaatgtggaatacctaaggatagcaccgcttttgttctcgcaaaaacgtatctcctaatttgaggctcagatgaaatgcatcaggtcctttggggcatagatcagaagactacaaaaatgaagctgctctgaaatctcctttagccatcaccccaaccccccaaaattagtttgtgttacttatggaagatagttttctccttttacttcacttcaaaagctttttactcaaagagtatatgttccctccaggtcagctgcccccaaaccccctccttacgctttgtcacacaaaaagtgtctctgccttgagtcatctattcaagcacttacagctctggccacaacagggcattttacaggtgcgaatgacagtagcattatgagtagtgtggaattcaggtagtaaatatgaaactagggtttgaaattgataatgctttcacaacatttgcagatgttttagaaggaaaaaagttccttcctaaaataatttctctacaattggaagattggaagattcagctagttaggagcccaccttttttcctaatctgtgtgtgccctgtaacctgactggttaacagcagtcctttgtaaacagtgttttaaactctcctagtcaatatccaccccatccaatttatcaaggaagaaatggttcagaaaatattttcagcctacagttatgttcagtcacacacacatacaaaatgttccttttgcttttaaagtaatttttgactcccagatcagtcagagcccctacagcattgttaagaaagtatttgatttttgtctcaatgaaaataaaactatattcatttccactctaaaaaaaaaaaaaaaaa

SEQ ID NO: 16 - protein, 1210 aa, Homo sapiens [sequence omitted]